(19) United States
(12) Patent Application Publication
(10) Pub. No.: US 2019/0125309 A1
(43) Pub. Date: May 2, 2019

(54) SYSTEMS AND METHODS FOR ESTIMATING CARDIAC STRAIN AND DISPLACEMENT USING ULTRASOUND

(71) Applicants: Duke University, Durham, NC (US); Aalborg University, Aalborg (DK)

(72) Inventors: Olaf Von Ramm, Durham, NC (US); Joseph Kisslo, Durham, NC (US); Cooper Moore, Durham, NC (US); Martin Vandborg Andersen, Aalborg (DK); Samuel Emil Schmidt, Aalborg (DK); Peter Sogaard, Kobenhavn (DK); Lasse Riis Ostergaard, Klarup (DK)

(21) Appl. No.: 16/094,683

(22) PCT Filed: Jun. 26, 2017
(86) PCT No.: PCT/US2017/039290

§ 371 Date: Oct. 18, 2018

Related U.S. Application Data
(60) Provisional application No. 62/354,432, filed on Jun. 24, 2016.

(30) Foreign Application Priority Data
Jul. 1, 2016 (DK) PA 2016 00394

(51) Int. Cl. A61B 8/08; A61B 8/00

(52) U.S. Cl. CPC A61B 8/485; A61B 8/0883; A61B 8/486; A61B 8/466; A61B 8/5276; A61B 8/463; A61B 8/5207

(57) ABSTRACT

Systems and methods for estimating cardiac strain and displacement using ultrasound are disclosed. According to an aspect, a method includes receiving frames of images acquired from a subject over a period of time. The method also includes determining, in the frames of images, speckles of features of the subject. Further, the method includes tracking bulk movement of the speckles in the frame of images. The method also includes determining displacement of at least a portion of the subject based on the tracked bulk movement.

### CROSS REFERENCE TO RELATED APPLICATIONS

This application claims priority to U.S. Provisional Patent Application No. 62/354,432, filed Jun. 24, 2016 and titled SYSTEMS AND METHODS FOR THE ESTIMATION OF CARDIAC STRAIN USING HIGH FRAME RATE ULTRASOUND, the disclosure of which is incorporated herein by reference in its entirety.

This application claims priority to Denmark Patent Application Number PA 2016 00394, filed Jul. 1, 2016, the disclosure of which is incorporated herein by reference in its entirety.

### TECHNICAL FIELD

The presently disclosed subject matter relates to imaging systems and method. More particularly, the presently disclosed subject matter relates to systems and methods for estimating cardiac strain and displacement using ultrasound.

### BACKGROUND

Cardiac disease is the leading cause of morbidity and mortality among humans over the age of 40. For example, heart failure is common in the United States, occurring in 6% to 10% of the adult population above the age of 65. Heart failure can be the result of either electrical or mechanical abnormalities. The effects of both mechanical and electrical abnormalities are difficult to distinguish in early stages of heart failure, making accurate diagnosis and effective treatment difficult.

Detection and measurement of rapid mechanical phenomena, such as the propagation of mechanical contraction and relaxation subsequent to depolarization, could have great clinical significance. Depolarization propagates with a velocity between 0.5 and 2 m/s depending on whether the electrical excitation is through the myocardial tissue or the Purkinje fibers. It is unlikely that these events can be detected using conventional ultrasound scanners, which operate at 50 to 110 frames per second (fps). A frame rate of greater than 500 fps is necessary to record information about mechanical activation sequences and correlate them to electrical measurements acquired by electrocardiography (EKG).

If these mechanical phenomena are similar in speed and duration to the propagation of the electrical excitation wave through the left ventricle, they will last 30 milliseconds. These rapid mechanical phenomena are currently undetected because of the low image acquisition rates of conventional clinical ultrasound imaging.

A common technique for automating measurements in ultrasound images is speckle or feature tracking. The most commonly used commercial speckle tracking algorithms, such as GE Healthcare's Automated Function Imaging software and TomTec's 2D (two-dimensional) Cardiac Performance Analysis software, use optical flow methods for frame-to-frame myocardial motion tracking. Optical flow has a lower limit of detectable velocities that depends on the algorithm's ability to detect sub-pixel variations between frames. If the movement of a target between two sequential images is smaller than that between adjoining spatial samples, and the tracking method does not implement sub-pixel variation detection, the frame-to-frame optical flow approach will not be able to detect the movement. Multiple studies on speckle tracking state that the best tracking accuracy is achieved using frame rates between 40 and 110 fps, as this gives the best trade-off between image quality and frame rate. This finding reflects the performance of currently available commercial diagnostic machines, which increase frame rate by reducing spatial sampling. Any measurement or observation derived from ultrasound images with inadequate spatial sampling may suffer from artifacts not indicative of actual myocardial motion. Currently there are no commercial diagnostic machines capable of capturing B-mode ultrasound images with the same temporal resolution as tissue Doppler imaging (TDI, greater than 180 fps) or ordinary EKG sampling rate (greater than 500 Hz) while maintaining adequate spatial sampling. If temporal resolution were to be increased without affecting spatial sampling, then clinicians would have a better tool to investigate myocardial motion and its relationship to electrical events in the heart.

One publication presented strain rate curves derived from TDI at 1200 samples per second. However, as TDI is a one-dimensional (1D) technique, it cannot discriminate between motion in different directions in the myocardium. Nevertheless, systolic and diastolic performance assessed using higher-temporal resolution TDI has provided additional information not available with conventional frame rate B-mode ultrasound imaging. Using TDI with a sample rate of 350 and 560 Hz, some publications described wave propagation in the inter-ventricular septum with spatial origins, temporal onsets and a propagation velocity similar to those of the electrical excitation from Purkinje fibers. Increasing temporal resolution of B-mode ultrasound images may therefore allow capture of these rapid events that otherwise would remain unappreciated. Several methods exist to increase the temporal resolution of ultrasound scanners without significantly decreasing spatial resolution, field of view (FOV) or depth. Retrospective gating is a method in which multiple ultrasound images recorded at high frame rate and narrow FOV are reconstructed into a single normal FOV image. However, prolonged acquisition time and movement artifacts cause the method to fail in many circumstances. Another method for increasing frame rate is multi-line transmit imaging, in which multiple focused beams are transmitted simultaneously, but artifacts occur because of crosstalk between the transmit beams. Widening the transmit beam by using an either unfocused or negatively focused transmit beam allows for the acquisition of multiple received image lines simultaneously, increasing frame rate. One publication presented live B-mode ultrasound images using a negatively focused transmit beam at up to 2500 fps for general applications and 1000 fps for images of the adult human heart. For a fixed amount of transmitted energy, an unfocused or negatively focused transmit beam will, however, have lower acoustic pressure across the image field than a focused transmit beam, resulting in lower signal to noise in the resulting images. Although derived strain curves from high-frame-rate B-mode ultrasound images (greater than 500 fps) were not found in the research of one publication, velocity curves from areas with strong myocardial signal in ultrasound sequences recorded at 900 fps have been derived. The researchers of that publication used coherent spatial compounding to reduce noise in their velocity measurements. This effectively reduced the frame rate to 180 fps. Coherent spatial compounding does have the potential to create high-quality ultrasound images at more than 300 fps. Another method for improving image quality is harmonic imaging that produce harmonics in the ultrasound field. A significant improvement in image quality with less clutter and better blood contrast can be achieved, but it requires high acoustic pressure, which can be an issue for phased array ultrasound systems and is severely range limited.

High-speed cardiac ultrasound images at frame rates above 100 fps have not been available in the art. Such high-speed images would allow for a more detailed assessment of cardiac motions, such as patterns of ventricular contraction, velocity of contraction, rate of valve openings and closings as well as timing interval information of various wall contractions. This improved temporal information may lead to improved diagnosis of pathologies such as degree of infarction, conduction disorders such as bundle branch blocks, and valve stiffness. Accordingly, it is desired to provide systems and methods that can permit strain and motion determinations from high speed 2D or three-dimensional (3D) ultrasound images.

### SUMMARY

Disclosed herein are systems and methods for estimating cardiac strain and displacement using ultrasound. According to an aspect, a method includes receiving frames of images acquired from a subject over a period of time. The method also includes determining, in the frames of images, speckles of features of the subject. Further, the method includes tracking bulk movement of the speckles between image frames. The method also includes determining displacement of at least a portion of the subject based on the tracked bulk movement.

According to another aspect, a method includes receiving frames of images acquired from a subject over a period of time. Further, the method includes qualifying, in the frames of images, speckles of features of the subject based on brightness. The method also includes tracking bulk movement of the qualified speckles between image frames. Further, the method includes determining displacement of at least a portion of the subject based on the tracked bulk movement.

### BRIEF DESCRIPTION OF THE DRAWINGS

The foregoing summary, as well as the following detailed description of various embodiments, is better understood when read in conjunction with the drawings provided herein. For the purposes of illustration, there is shown in the drawings exemplary embodiments; however, the presently disclosed subject matter is not limited to the specific methods and instrumentalities disclosed.

FIG. 1 is an image depicting an example coloring scheme of strain curves in accordance with embodiments of the present disclosure;

FIG. 2 is an image of an example myocardial region with 21 features tracks in accordance with embodiments of the present disclosure;

FIG. 3A is a graph showing average lateral tracking error as a function of translation increments after 10 mm of total translation;

FIG. 3B is a graph showing range tracking error as a function of translation increments after 10 mm of total translation;

FIGS. 4A and 4B are graphs showing tracked motion as a function of actual motion at different distances from the transducer for lateral translation;

FIGS. 4C and 4D are graphs showing tracked motion as a function of actual motion at different distances from the transducer for range translation;

FIGS. 5A-5F are graphs showing beat to beat comparison of the strain curves of a patient;

FIG. 6 is a graph showing an example of a MSW strain curve as a function of time seen as the black curve with the simultaneously recorded EKG signal seen in green immediately before and after isovolumetric contraction of the left ventricle;

FIGS. 7A and 7B are graphs showing circumferential strain curves derived from ultrasound images using the PSAX view from a 71 year old male with LBBB and a biventricular (BiV) pacer; and

FIGS. 8A and 8B depict a flow diagram of an example method for estimating cardiac strain and displacement in accordance with embodiments of the present disclosure.

### DETAILED DESCRIPTION

The presently disclosed subject matter is described with specificity to meet statutory requirements. However, the description itself is not intended to limit the scope of this patent. Rather, the inventors have contemplated that the claimed subject matter might also be embodied in other ways, to include different steps or elements similar to the ones described in this document, in conjunction with other present or future technologies. Moreover, although the term “step” may be used herein to connote different aspects of methods employed, the term should not be interpreted as implying any particular order among or between various steps herein disclosed unless and except when the order of individual steps is explicitly described.

Articles “a” and “an” are used herein to refer to one or to more than one (i.e. at least one) of the grammatical object of the article. By way of example, “an element” means at least one element and can include more than one element.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25 26. 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2% 1%, 0.5%, 0.1%, 0.05%, or 0.01% of the stated value. Unless otherwise clear from context, all numerical values provided herein are modified by the term about

Unless otherwise defined, all technical terms used herein have the same meaning as commonly understood by one of ordinary skill in the art to which this disclosure belongs.

In accordance with embodiments, the presently disclosed subject matter relates to systems and methods for speckle feature tracking and strain estimation in high-frame-rate B-mode ultrasound images acquired using a phased array ultrasound scanner. In vitro validation of the tracking algorithm was performed and applied to patient studies.

Image acquisition rate in ultrasound is limited by the number of transmit-receive (Tx-Rx) operations and the speed of sound in tissue. Parallelism in receive, also known as explososcanning, can increase frame rate while maintaining adequate spatial sampling. In a study described herein used ultrasound images acquired using Duke University's phased array ultrasound scanner, referred to as “T5”. A 96-element, 1D phased array (Volumetrics, Durham, N.C., USA) was used for image acquisition. The active aperture of this array was 21 mm in azimuth and 14 mm in elevation. For all images in the study, a two-cycle, 3.5-MHz transmit pulse was used. The theoretical diffraction-limited resolution cell was 1.2 degree 0.44 mm. Resulting ultrasound images had 160 receive image lines with 0.5 degree×0.25 mm sampling (xₛ) for this configuration, giving a 80 degree FOV and a scan depth of up to 140 mm. For this study, T5 was programmed to use 16:1 parallel receive processing to maintain close to uniform beam strength across the scan. As a result, frame rates were limited to 500 fps for adult echocardiograms. In this study, no compounding, apodization or harmonic imaging techniques were employed.

In accordance with embodiments, a framework for a continuous speckle feature tracking (CSFT) algorithm using features derived from speckle is disclosed. The CSFT algorithm may function with high-frame-rate ultrasound images and, therefore, was effective for tracking small inter-frame translations. The CSFT algorithm pipeline in examples described herein include five independent steps or processing blocks. Each step in the pipeline can be manipulated without altering other blocks in the pipeline. It is noted that in this example the CSFT algorithm or method is described as being applied to frames of images acquired from a heart of a subject over a period of time, although it should be understood that the algorithm or method may otherwise be suitably applied to any subject.

Initially, the CSFT algorithm may include a pre-processing step. The pre-processing step may include image filtering. For example, image filtering may be in both the temporal and spatial domains to reduce noise present in the ultrasound images and improve feature extraction. A temporal bandpass filter may be used to dampen high-frequency noise and image artifacts. As blood flow velocities within the ventricle are typically higher than those of myocardial contraction, tracking blood can contribute noise in motion tracking and can therefore be filtered. Because the normal resting heart rate of an adult is 60 bpm, the lower cutoff of this example filter was set at 1 Hz. The temporal filter may be a Hamming-window bandpass between 1 and 250 Hz. A spatial filter may be used to blur individual frames, as the smooth texture improved feature extraction. For this, a Gaussian filter may be applied in the native polar coordinates. The size of the spatial filter kernel (W2D) was 3×xₛ in this example to encompass one resolution cell.

In a step subsequent to the pre-processing step, features may be extracted from the pre-processed images. For example, features used in this algorithm may be derived from speckle in the acquired frames of images, such as B-mode images. In accordance with embodiments, contour through the middle of the ventricular myocardium may be drawn manually. B points (O=O₁, . . . , O_{b}, . . . , O_{B}) may be automatically defined equally distributed along the contour. For the realization of the algorithm, we set B at 17. A region of interest (ROI_{b}) equal to two times the thickest part of the myocardium was centered around each point O_{b}, where O_{b} is illustrated as the center of the circles shown in FIG. 1, which is an image depicting an example coloring scheme of strain curves. (1) the basal septal wall (BSW), (2) for the mid-septal wall (MSW), (3) the apical septal wall (ASW), (4) the apical lateral wall (ALW), (5) the mid-lateral wall (MLW), (6) the basal lateral wall (BLW). The thickness of these regions was defined by manually drawing a line across the thickest part of the interventricular septum, illustrated by the double-headed arrow shown in FIG. 1. Features were identified by two criteria. First, the intensity (I_{i,n}) of pixel i in frame n had to be an automatic computed threshold for a region, which can be calculated using etc. the Otsu algorithm. Otsu is a method of automatically finding a threshold. Second, I_{i,n} had to be larger than all pixel intensities in a W_{2D} neighborhood. Within region ROI_{b} all speckle center coordinates (Pₙ) and their corresponding intensities Iₙ were stored. Features outside ROI_{b} may be excluded.

Subsequent to feature extraction, features may be tracked. In an example of feature tracking, an optimization algorithm may be used to link speckle features between frames for as long as each speckle is visible. Because the Euclidean distance was used as a translation threshold, all points Pₙ may be converted to Cartesian coordinates. Limitations can be set on the translation and intensity change to remove outliers that were not likely to be the same speckle. The maximum translation of heart tissue between frames (dᵣ,fₛ) may be defined by using physiologic constraints. Maximum velocities of myocardial tissue may be derived in both the long and short axes in healthy adults using TDI. On the assumption that peak velocities in both long and short axes occurred simultaneously, dᵣ,fₛ was defined as

$${d}_{r , {f}_{s}} = \frac{221 \frac{\begin{matrix}m \\ m\end{matrix}}{s}}{{f}_{s}} \qquad ( 1 )$$

where fₛ is the frame rate. The smallest detectable increment for this algorithm is xₛ. Given the high frame rate, tissue may move less than one spatial image sample between frames, necessitating tracking over longer time records than sequential frames.

As any given ROI could contain multiple features, an optimization algorithm may be used to determine frame-to-frame links. A suitable combinatorial optimization algorithm may be used to find a globally optimized combination within a finite set. Two features may be used to describe the relationship between potential feature links. The Euclidean distance may be calculated between “features in frame n” and “features in frame n,” giving a distance matrix (D_{n-1,n}). The absolute logarithmic value of the intensity ratio was calculated between “Iₙ₋₁ in frame n−1” and “Iₙ in frame n,” giving an intensity ratio matrix (IR_{n-1,n}). To ensure that the same features were tracked, a threshold (dₘₐₓ) equal to d_{r,fs}*125% was set to ensure that features outside the maximal feasible translation distance were not evaluated as possible links. It was assumed that there would not be a large change in intensity of features between frames. If was greater than 6 dB, features may not be linked. A suitable algorithm may be implemented to calculate links between features using a cost function combining the two matrices D_{n-1,n} and IR_{n-1,n}.

In another subsequent step, the CSFT method may implement region tracking. Even in the perfect scenario with no out-of-plane motion speckle, and, by extension, features can only be tracked for 40% of the aperture width due to speckle decorrelation, which corresponded to a distance of 8 mm for the 21 mm transducer used. To remove discontinuities a moving point (X(n)) representative of the average movement of multiple features in a region was calculated. Thereby making continuous tracking of local myocardial motion throughout the entire cardiac cycle possible without using predefined models or other supervised learning methods.

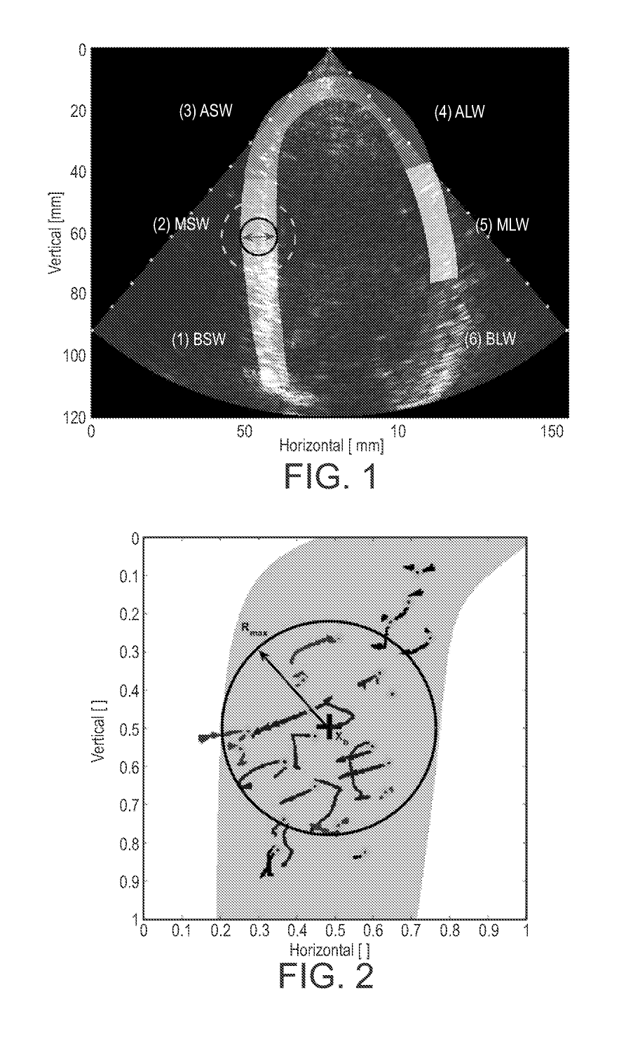

A qualification of tracked motion was used to discard tracked features moving in a different direction compared to the bulk of myocardial motion. To limit the search to features in the myocardium, a radius (Rₘₐₓ) equal to half the myocardial thickness from X_{b}(n) was defined and illustrated by the black circle on FIG. 1 and the black arrow and circle on FIG. 2. FIG. 2 illustrates a myocardial region with 21 features tracks. The gray area illustrates the myocardium, the black, blue and red curves describe features tracks. The currently active frame on each of the feature tracks are illustrated by the green circles. If feature track i was outside the radius Rₘₐₓ in frame n it was denoted as inactive and illustrated as black in FIG. 2. If feature i was tracked was inside Rₘₐₓ, the entire feature track (Tᵢ(n)) was denoted as active which is illustrated as blue and red curves in FIG. 2. A similarity measure (Eᵥ) was defined by Equation 2 below.

$${E}_{v} ( j ) = {\sum }_{i = 1 , j \neq i}^{K} {\sum }_{n = 1}^{{N}_{i , j} - 1} \frac{\vert {T}_{i}^{′} ( n ) - {T}_{j}^{′} ( n ) \vert}{{N}_{i , j}} \qquad ( 2 )$$

where K is the number of active tracks within a distance Rₘₐₓ of the point X_{b}(n), N_{i,j} was the number of frames where the active tracks i and j were simultaneously active, |T′ᵢ(n)−T′ⱼ(n)| denotes the Euclidean distance and T′ᵢ(n) is the derivative of track Tᵢ(n). The velocity (V_{b}(n)) was calculated by fitting a spline curve to the derivative of the active tracks where Eᵥ was below an Otsu threshold, illustrated by the blue curves in FIG. 2. The bulk displacement (Y_{b}(n)) was then given by the time integral of V_{b}(n).

FIG. 2 shows an example of feature tracks inside and outside a radius (Rₘₐₓ) from the center (X_{b}) of a myocardial segment in the interventricular septum, which is illustrated by the black cross. The black arrow is the length of the Rₘₐₓ, while the black circle describe the entire border of Rₘₐₓ. The current frame being evaluated is illustrated using a green circle on each of the feature tracks. If a feature track was outside Rₘₐₓ was defined as inactive, which is illustrated using a black curve with directional arrows. Feature tracks inside Rₘₐₓ and the corresponding similarity measure (Eᵥ) below the Otsu threshold may be considered a strong feature track and may be used for calculating the velocity of the segment (V_{b}(n)). Feature tracks inside Rₘₐₓ and the corresponding Eᵥ was above the Otsu threshold were considered weak and not used for the calculation of V_{b}(n), which is shown by the curves with directional arrows.

It was assumed that the heart would return to its original position at the end of each cardiac cycle. The possible drift component (Cb,drift(n)) can be removed, as Yb(n) may have a baseline drift caused by tracking errors, patient or operator motion or respiratory changes. Cb,drift(n) was calculated as the piecewise linear change in coordinates of Yb(n) between each Q wave onset (tQ), as noted from the EKG. By subtracting the component from each position during the cardiac cycle, the drift compensated position vector Xb(n) was estimated.

In a subsequent step, strain may be calculated. For example, longitudinal cardiac strain (ε(n)) may be calculated from the B points along the myocardial contour given by Xb(n). Strain represents the fractional heart wall deformation through time with respect to an initial condition and is a metric that is independent of the orientation and size of the ventricle.

In experiments, tracking accuracy of the CSFT algorithm was validated in vitro. A tissue mimicking phantom (CIRS Model 040GSE Multi-Purpose Multi-Tissue Ultrasound Phantom, Computerized Imaging Reference Systems, Norfolk, Va., USA) was imaged using the T5 system. The scanner was programmed to acquire a single B-mode image when an external manual trigger button was pressed. The transmit beam was focused at 60 mm, and only one image line was received for each transmit as in conventional scanning. The 3.5-MHz, 96-element 1-D array was clamped to a high-resolution translation stage and moved manually in 10-micrometer increments. At each location, a B-mode image was recorded. Two sets of ultrasound images were acquired to mimic pure lateral or pure range translations. A sequence of 1000 frames was acquired with 10 micrometer translations between image frames for both lateral and range translations, for a total translation of 10 mm in each set. The phantom contained specular targets as well as regions of speckle. Nine ROIs were selected manually to ensure that specular targets were in all frames. The effective velocity that these translations represent depends on the frame rate. For example, with a frame rate of 500 fps, a 10-mm translation would correspond to a velocity of 5 mm/s, and a 1-mm translation increment would correspond to an effective velocity of 0.5 m/s. The effective velocity being tracked was increased by tracking between non-sequential frames up to 1.00-mm displacements. For the CSFT algorithm, dₘₐₓ was manually set to 1.5 mm for all simulated velocities to ensure that all the tested increments can be tracked. For the translation measurements, the linear baseline drift compensation in the CSFT algorithm was disabled. Accuracy was measured using a tracking error between the measured translation and tracked translation at different translation velocities using sequential and non-sequential images from the translation data. In vivo four-chamber strain curves. Strain curves derived from high-frame-rate images using the CSFT algorithm were compared in 10 patients (6 males and 4 females, average age 5 62, range: 27-81), both with and without cardiac disorders (6 patients without cardiac abnormalities, defined as QRS duration below 90 ms, no left ventricular contraction abnormalities and normal ejection fraction). A clinician scanned each patient to acquire apical four-chamber (AP4) images with the T5 system. An expert used the CSFT algorithm to estimate strain curves in two to five cardiac cycles from the T5 ultrasound images to assess beat-to-beat reproducibility. Strain curves were derived from the AP4 images at six standardized locations in the inter-ventricular septum and lateral wall using the CSFT algorithm. Myocardial segment color and names were assigned to the six curves with respect to their position. These segments were: basal septal wall (BSW, green), mid-septal wall (MSW, light purple), apical septal wall (ASW, light blue), apical lateral wall (ALW, blue), mid-lateral wall (MLW, yellow) and basal lateral wall (BLW, purple). These portions are shown in grayscale, not color, in FIG. 1. Fisher's z-transformed average correlation coefficient (r_{z′}) was calculated between all beats and patients to test the CSFT algorithm's in vivo beat-to-beat reproducibility of strain curves. r_{z′} was used because it has less bias when estimating a population correlation compared with the averaged correlation coefficient. r_{z′} was calculated by combining the Pearson correlation coefficient r and Fisher's z₀ transformation. Some variations were present between cardiac cycles. However, the entire R-R interval varies significantly more in a patient compared with the QT interval. The correlation coefficient was therefore calculated during the duration of the longest QT interval between all cardiac cycles in each individual patient.

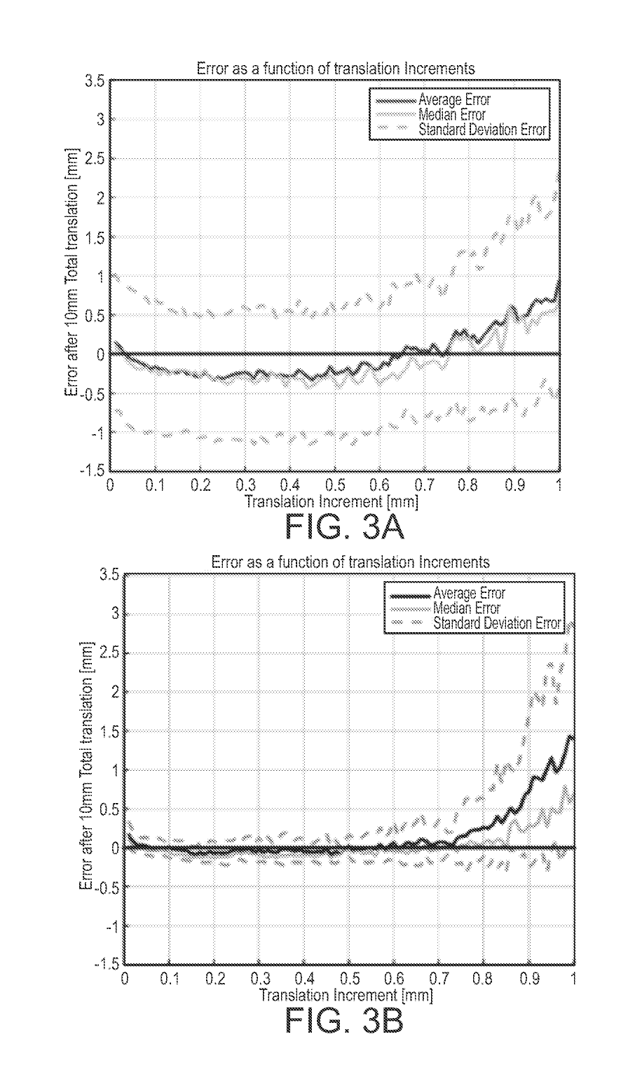

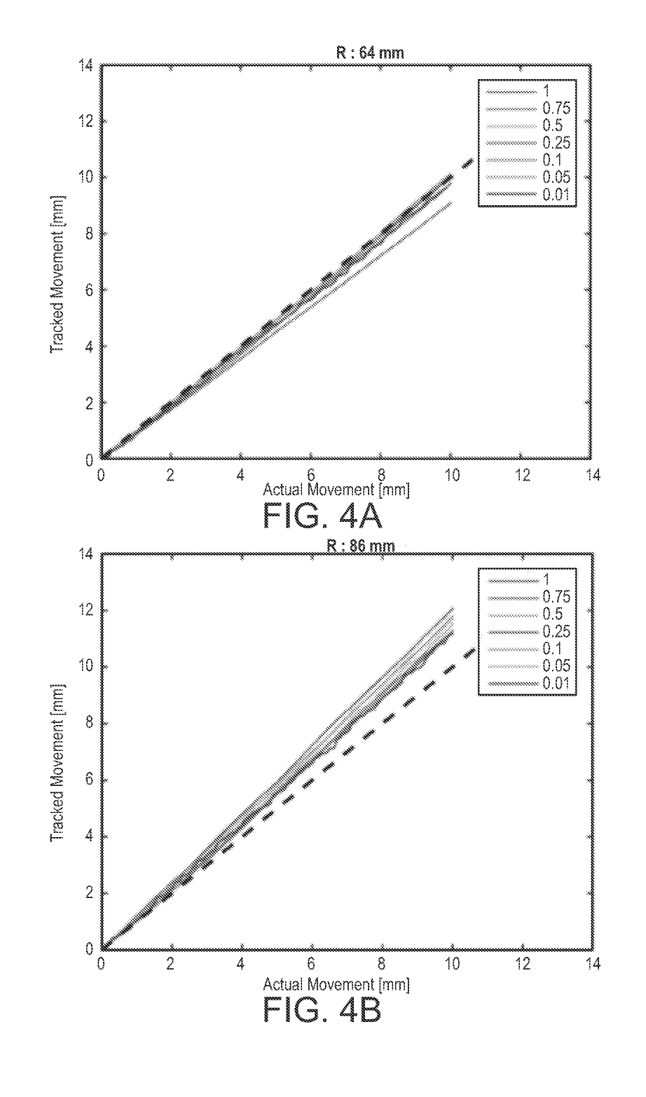

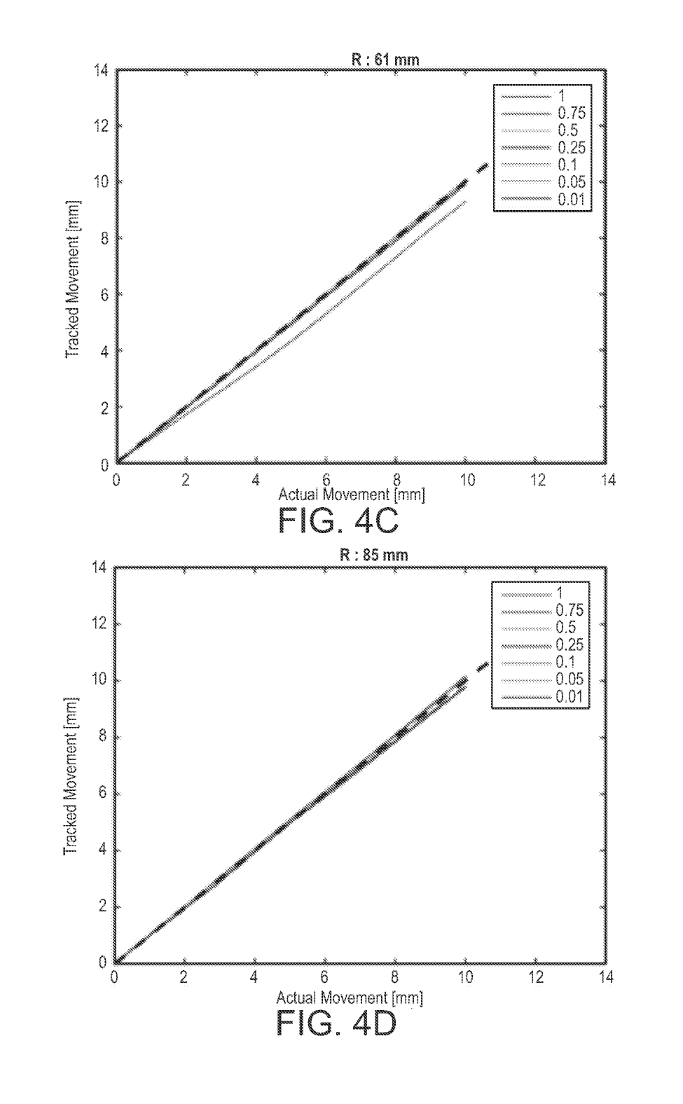

In the two tracking data sets, regions of speckle within a phantom were tracked over 10 mm of total translation, either in range or laterally, at nine different ranges from the transducer between 38 and 86 mm. The CSFT algorithm estimated translation for different translation increments between 10 mm and 1 mm in 10-micrometer steps, and results were compared with the actual translation between 0 and 10 mm. The increments correspond to velocities of 5 to 500 mm/s when acquiring images at 500 fps. The tracking results of the CSFT algorithm for lateral and range translations are presented in FIGS. 3A and 3B, in which the measured translation is presented on the xaxis, and the tracked motion per the CSFT algorithm, on the y-axis. The curves represent different increments of actual translation between frames, ranging from 0.01- to 1.0-mm steps. Each figure illustrates the results of the CSFT algorithm at a different depth in the image. The CSFT algorithm estimation was most accurate at the focus of the transmit beam, whereas a translation underestimation was present for ranges proximal to the transmit focus. For range translations close to the transducer, the CSFT algorithm estimated the translation to be 9.5±1.1 mm when the actual translation was 10.0 mm at a range of 39 mm. The CSFT algorithm estimated the translation to be 10.0±0.2 mm when the actual translation was 10.0 mm at a range of 50 mm. Proximal to the transmit focus, lateral motion was estimated to move more slowly than the actual motion. At a range of 38 mm, the estimated translation was 8.2±0.5 mm after 10 mm of actual translation. Distal to the transmit focus, lateral motion was estimated to move faster than the actual motion. For example, the best tracking accuracy was found near the focus of the transmit beam: at a range of 60 mm the estimated translation was 9.9±0.1 mm after 10 mm of actual translation (see FIG. 4C). At a range of 86 mm, the estimated translation was 11.2±0.3 mm after 10 mm of actual translation (see FIG. 4D). The average tracking error for the nine different scan depths was calculated after 10 mm of actual translation. FIGS. 3A and 3B illustrate the average error after 10 mm of actual translation as a function of individual translation increments for 0.01- to 1.00-mm actual inter-frame translation increments. The red curves represent average error, the cyan curves represent median error and the green curves represent standard deviation from the average error. For range translations in FIG. 3A, the average error was less than 10% for all tested translation increments. The standard deviation severely worsened when individual translation increments were larger than 0.8 mm/frame. For lateral translations in FIG. 3B, both the average error and standard deviation remained constant for all tested translation increments. Tracking error for range and lateral translation worsened as inter-frame translation increments increased.

In the two tracking datasets, regions of speckle within a phantom were tracked over 10 mm of total translation, either in range or laterally, at 9 different ranges from the transducer between 38 mm-86 mm. The CSFT algorithm estimated translation for different translation increments between 10 μm-1 mm in 10 μm steps and results were compared to the actual translation between 0 mm-10 mm. The increments correspond to velocities of 5 mm/s-500 mm/s when acquiring images at 500 fps. The tracking results of the CSFT algorithm for lateral and range translations are presented in FIGS. 4A-4D. In the figures, the measured translation is presented on the x-axis and the tracked motion per the CSFT algorithm on the y-axis. The curves represent different increments of actual translation between frames, ranging from 0.01 mm to 1.0 mm steps. Each figure display the results of the CSFT algorithm at a different depth in the image. The CSFT algorithm estimation was most accurate at the focus of the transmit beam, while a translation underestimation was present for ranges proximal to the transmit focus. For range translations close to the transducer, the CSFT algorithm estimated the translation to be 9.5±1.1 mm when the actual translation was 10.0 mm at a range of 39 mm. The CSFT algorithm estimated the translation to be 10.0±0.2 mm when the actual translation was 10.0 mm at a range of 50 mm. Proximal to the transmit focus, lateral motion was estimated to move slower than the actual motion. At a range of 38 mm the estimated translation was 8.2±0.5 mm after 10 mm of actual translation. Distal to the transmit focus, lateral motion was estimated to move faster than the actual motion. For example, the best tracking accuracy was found near the focus of the transmit beam, at a range of 60 mm the estimated translation was 9.9±0.1 mm after 10 mm of actual translation, see FIG. 3C. At a range of 86 mm the estimated translation was 11.2±0.3 mm after 10 mm actual translation, see FIG. 3D. The average tracking error for the 9 different scan depths was calculated after 10 mm of actual translation. FIGS. 4A and 4B show the average error after 10 mm of actual translation as a function of individual translation increments for 0.01 mm-1.00 mm actual inter-frame translation increments. The red curves represent average error, the cyan curves represent median error, and the green curves represent standard deviation from the average error. For range translations in FIG. 4A, the average error was less than 10% for all tested translation increments. The standard deviation severely worsened when individual translation increments were larger than 0.8 mm/frame. For lateral translations in FIG. 4B both the average error and standard deviation remained constant for all tested translation increments. Tracking error for range and lateral translation worsened as inter-frame translation increments increased.

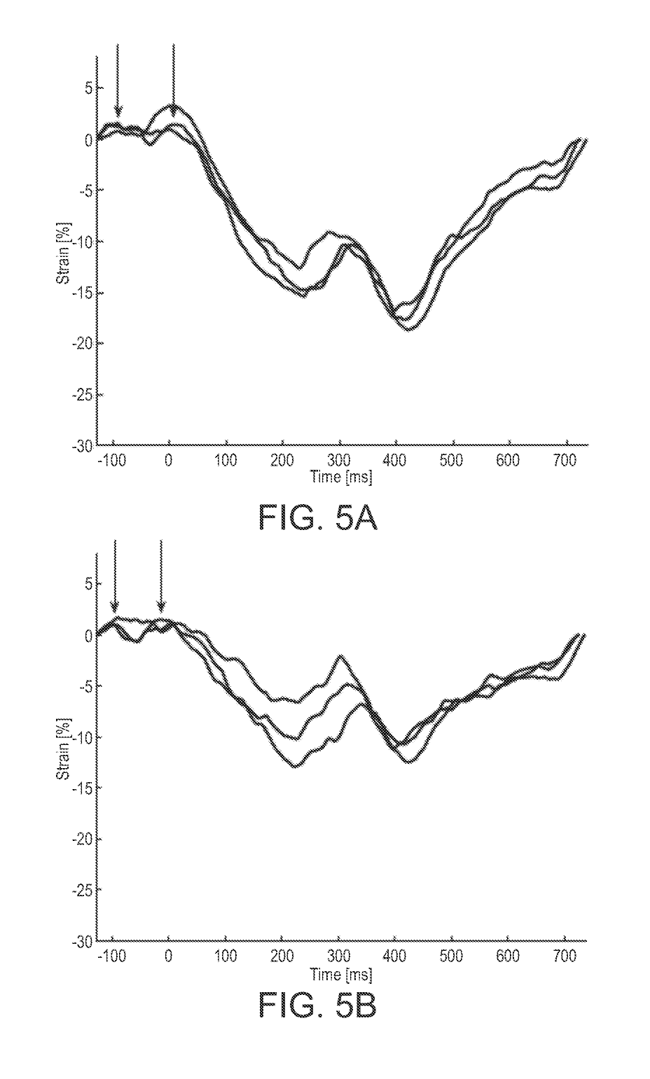

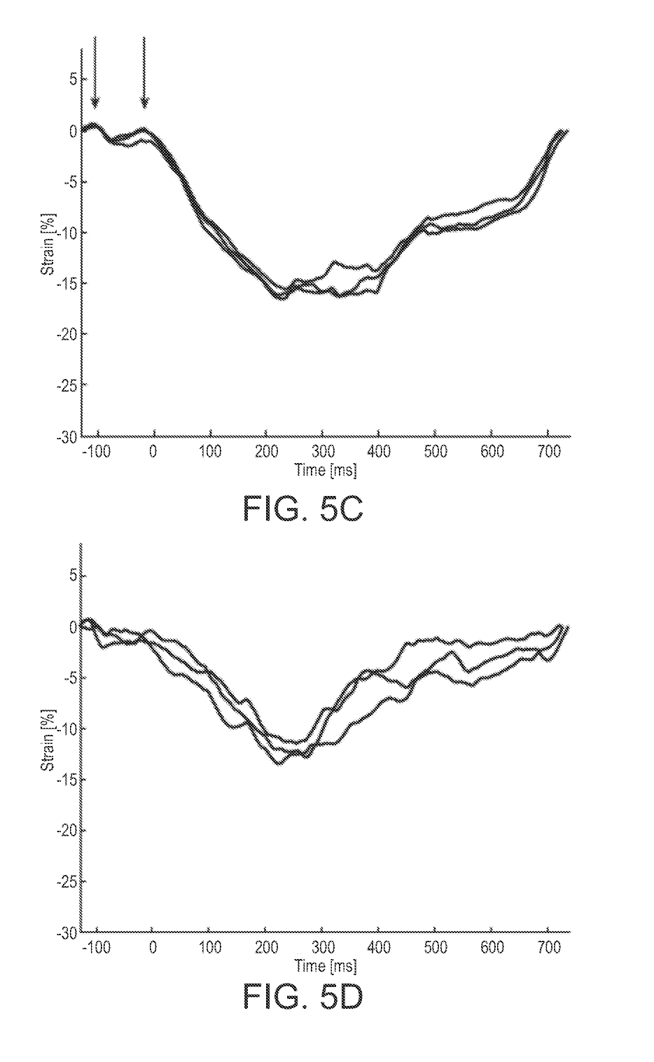

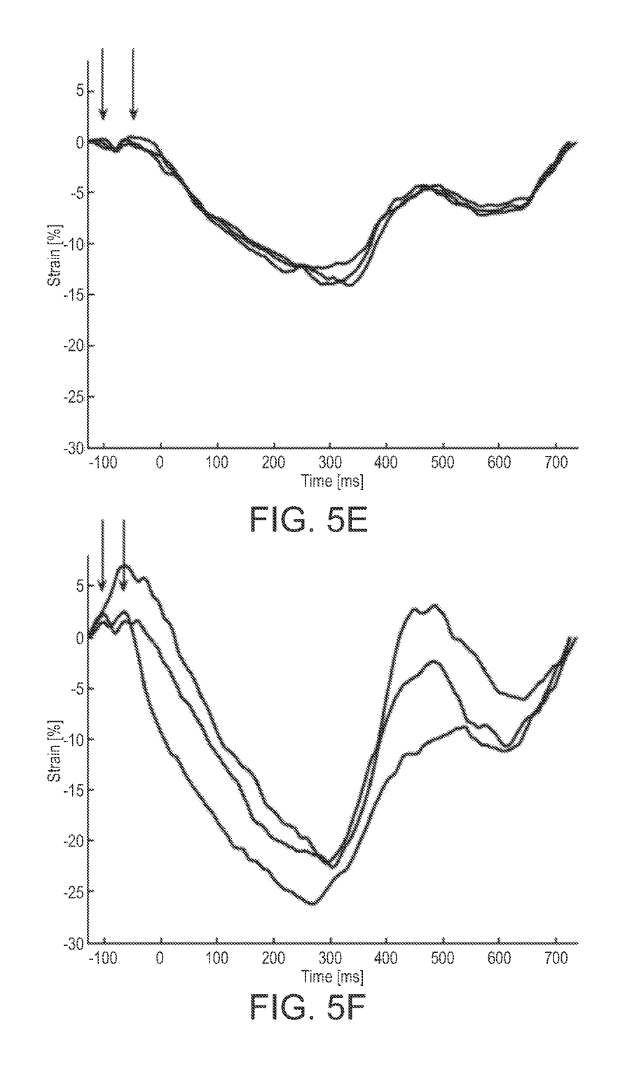

A clinical expert compared the beat to beat variations of strain curves generated by the CSFT algorithm using high frame rate ultrasound images. FIGS. 5A-5F show beat to beat comparison of the strain curves of Patient P010. Coloring of the strain curves were done with respect to the coloring scheme in FIG. 1. τ was set as the time point for baseline drift compensation.

The CSFT algorithm estimated six strain curves on two to five consecutive cardiac cycles. When high frequency content was caused by noise the entire curve was affected obscuring contractile information. Filtering the CSFT strain curves could have improved the appearance of the strain curves. However, high frequency information of potential clinical significance. For this reason the raw strain curves were presented. The mid lateral wall was difficult to track, due to shadowing from the lung as seen in the mid lateral wall illustrated in yellow on FIGS. 5A-5F. Beat to beat reproducibility of the strain estimation was measured using the Pearson correlation coefficient for each patient. The averaged correlation coefficient was calculated as r_{Z}′ between all patients. The resulting average correlation r_{Z}′ was estimated to 0.95 for the entire myocardial wall and the strain curves being significantly correlated in 97% of cases, see Table 1. A higher r_{Z}′ were calculated in the interventricular septum compared to the lateral wall.

Clinical ultrasound scanners today are hardware-limited, only allowing for increased temporal resolution by reducing spatial sampling or scan depth. A method for parallel receive processing, called explososcaning, that can increase temporal sampling rates has been used. While expanding the transmit beam will decrease resolution in transmit, the overall system resolution is the product of the Tx and Rx beam responses. Other studies have presented strain and strain rate derived from TDI acquired at up to 1200 samples per second that show useful information, not necessarily appreciated in conventional ultrasound images. However, Both M-mode and TDI are inherently one-dimensional techniques. High frame rate B-mode ultrasound imaging provides the opportunity for capturing the motion detail in two or three dimensions. Velocity curves at 180 to 900 fps in selective myocardial segments using B-mode imaging have been presented. This shows that the advent of high frame rate B-mode cardiac ultrasound imaging may permit the assessment of point to point myocardial contractions at temporal resolution commensurate with EKG sampling. The algorithm presented in this study was tailored to high frame rate ultrasound images and was designed to track small inter-frame translations of less than 1.00 mm. This ability to track small displacements permits distinguishing between the local myocardial contraction and the bulk movement of the heart. Also, the CSFT algorithm enables tracking of small sub resolution translations.

A fundamental assumption of speckle and feature tracking is that the speckle moves at the same velocity as the object being imaged. In the in vitro validation presented here, speckle proximal to the transmit focus were consistently estimated to move slower giving a total estimated translation of 8.2±0.5 mm and 9.5±1.1 mm for range and lateral translations respectively, compared to the 10 mm of actual translation. Speckle distal to the transmit focus was estimated to move faster than the objects' actual movement, with the CSFT algorithm estimating 10.0±0.2 mm and 11.2±0.3 mm of range and lateral translation respectively compared to the 10 mm of actual translation. The tracking errors at different inter-frame displacements are presented in FIGS. 4A-4D, which show both lateral and range tracking errors as a function of translation increments. It is clearly shown that even at increments below xₛ tracking error remains low. The CSFT algorithm performed better at estimating range translations than lateral translations.

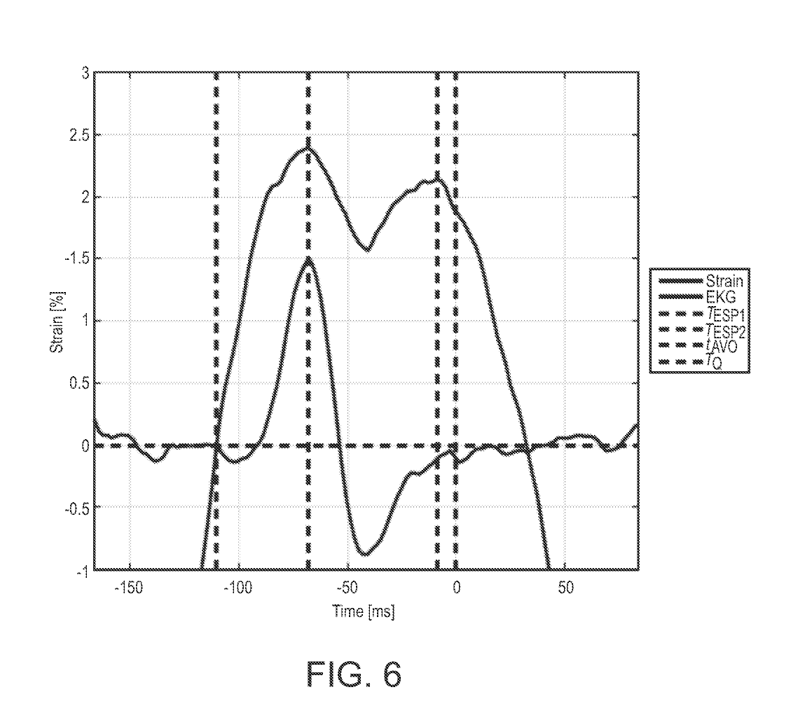

High frequency information was often obscured if a segment had low myocardial signal. A strongly reflective pericardium precluded tracking lower intensity signals in the myocardium as displayed in FIG. 5D. These regions of low myocardial signal were inconsistently tracked over multiple cardiac cycles, resulting in a lower correlation coefficient than in regions of high signal. When viewing ultrasound images recorded at 250 fps or greater at a reduced playback rate, rapid changes in individual speckle appearance and gross changes in the overall myocardial speckle patterns were observed prior to the onset of both contraction and relaxation. This phenomenon was seen across most patients and views. One potential origin of this phenomenon could be out of plane motion from muscle fibers contracting or relaxing. To further investigate this, it may be necessary to obtain high frame rate strain measurements in three dimensions. The isovolumetric contraction was noted to have two separate phases (ESP 1 and ESP 2), that were not observed in traditional low frame rate strain curves. These could be observed visually by viewing the high frame rate ultrasound sequences at 10% playback speed. FIG. 6 is an example of a MSW strain curve as a function of time seen as the black curve with the simultaneously recorded EKG signal seen in green immediately before and after isovolumetric contraction of the left ventricle. Aortic valve opening timing was defined using post hoc M-mode derived from the center scan line of the original B-mode ultrasound images and used as the global time reference. While these mechanical phenomena occur during isovolumetric contraction, their origin still warrants further investigation.

The CSFT algorithm presented here is a robust method for deriving myocardial strain curves from high frame rate B-mode ultrasound images. Though the algorithm did not implement image interpolation, it was capable of tracking motion of less than one resolution cell per frame. High frequency information in the strain curves derived from high frame rate ultrasound images was observed. This information was independent of overall cardiac motion. Tracking error was seen to increase for both range and lateral translation as a function of translational increments, to be interpreted as the analog to reducing frame rate sampling. This study also revealed that tracking accuracy varies not only as a function of frame rate but also of beam shape. In summary, increasing temporal resolution has the potential to allow for a more detailed description of myocardial contraction and its relationship to overall cardiac health.

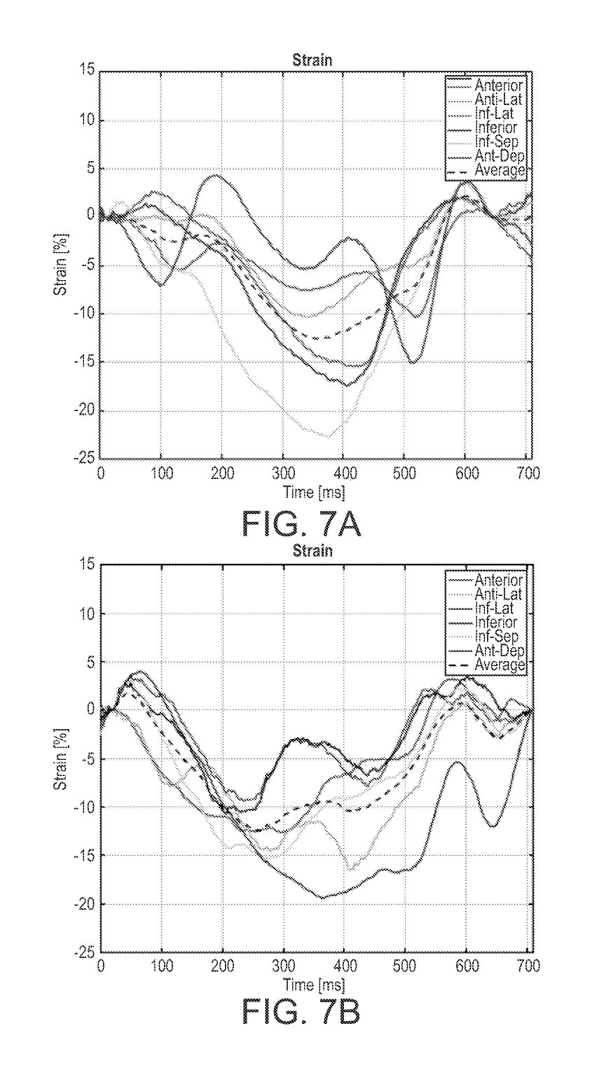

FIGS. 7A and 7B are graphs showing circumferential strain curves derived from ultrasound images using the PSAX view from a 71 year old male with LBBB and a biventricular (BiV) pacer. Ultrasound images were acquired on the same day and show how mechanical contraction differs between the BiV pacer being turned off and on respectively.

A measure from maximum myocardial stretching to end of the plateau immediately before mechanical contraction onset (tₒₙₛₑₜ) and the first minimum nadir after mechanical contraction onset (t_{contract}) was manually measured, see the following table.

TABLE
Timing Results

| | tₒₙₛₑₜ [ms] | T_{contract} [ms] | tₒₙₛₑₜ [ms] − t_{contract} [ms] |
|---|---|---|---|
| BiV_{off} | 89 +/− 69 | 312 +/− 144 | 222 |
| BiVₒₙ | −9 +/− 20 | 211 +/− 55 | 220 |

FIG. 7A show that the myocardial segments do not synchronously contact when the BiV pacer off, and that the BiV pacer on the mechanical contraction occurs more synchronously, see FIG. 7B. The average time between tₒₙₛₑₜ and t_{contract} only differed by 4 ms, see the above table. Furthermore, both tₒₙₛₑₜ and t_{contract} with the BiV pacer off had a higher variation than with the BiV pacer on, see the above table.

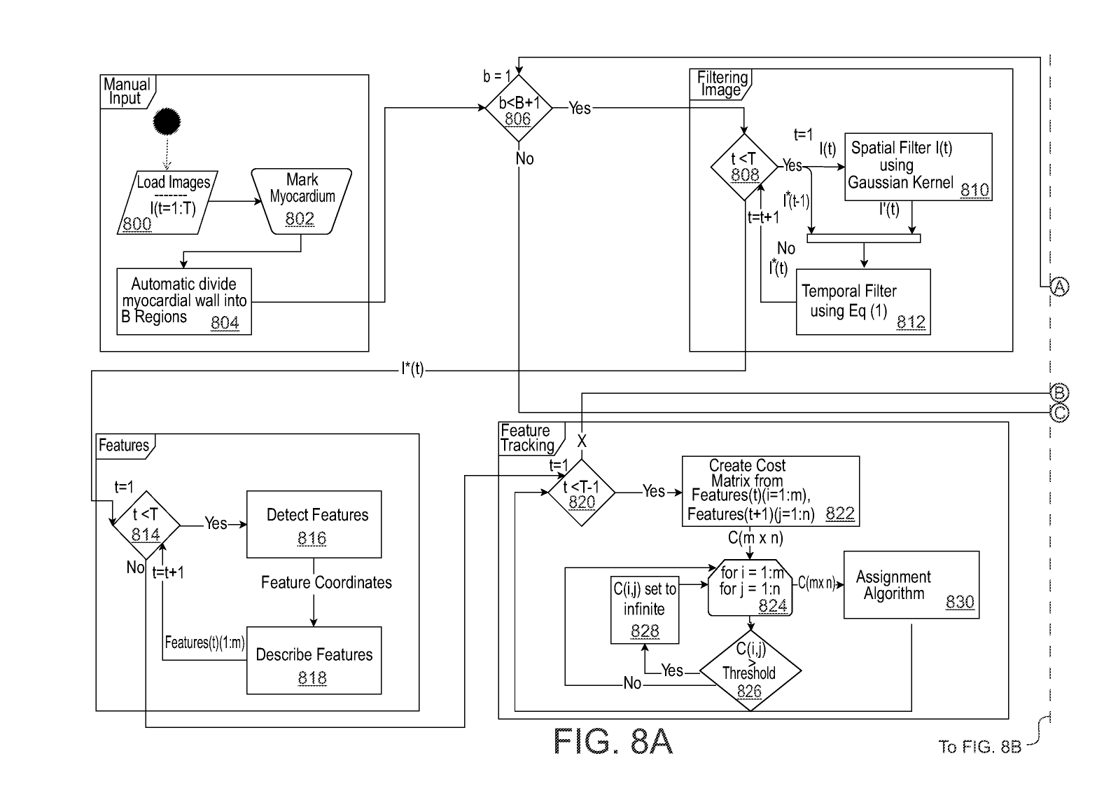

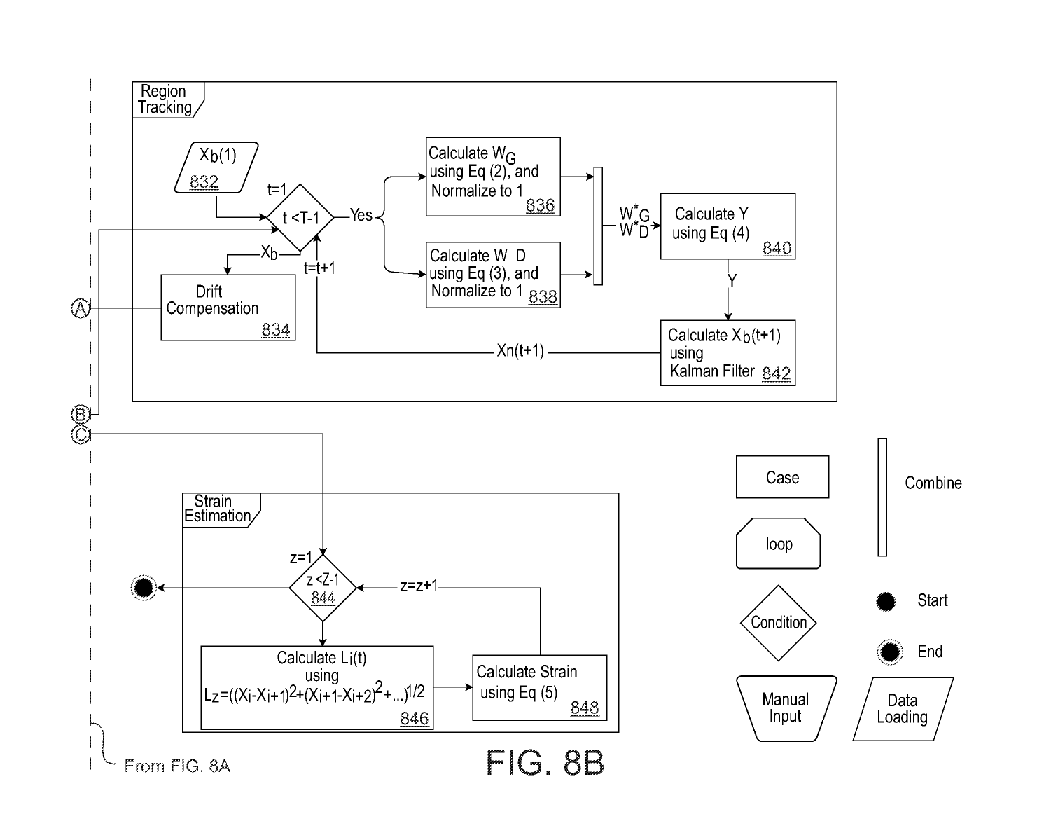

FIGS. 8A and 8B depict a flow diagram of an example method for estimating cardiac strain and displacement in accordance with embodiments of the present disclosure. The method may be implemented by any suitable computing device capable of processing image data, analyzing image data, and presenting analysis results to a user. For example, the computing device may be operably connected to an ultrasound system for receipt of frames of ultrasound images of a heart. Although in this example, the method is described as being applied to capture of ultrasound images of a person's heart. It should be understood that the method may otherwise be suitably applied for estimating or determining displacement or velocity of any other subject based on any suitable type of acquired images.

Referring to FIGS. 8A and 8B, the method may begin by loading 800 images into a computing device. The images in this example are frames of ultrasound images acquired over a period of time. The ultrasound images may be 2D ultrasound images, 3D ultrasound images, or any other suitable types of images.

The method of FIGS. 8A and 8B also include marking 802 myocardium. For example, the mid myocardium may be marked along a contour and the thickness region in the myocardium (L_{T}). The myocardium can be marked in accordance with other examples disclosed herein. The method also includes automatically dividing 804 the myocardial wall into B regions. Step 802 includes marking the myocardial contour line, and width of the myocardium in such a way, so information about the width and location of the myocardial wall at any point along the myocardial contour is provided. At step 804, the myocardial contour line may be used to evenly divide the contour into B point which will be the center of the B+1 regions. Steps 800, 802, and 804 are part of a manual input portion of the method.

The method of FIGS. 8A and 8B further include determining whether b is greater than B+1 at decision step 806. At step 806: b is set to 1, and B is set to an arbitrary number of regions from which to calculate motion form along the myocardial contour, such as B=19. If “Yes” at step 806, the method proceeds to filter images. Image filtering includes steps 808, 810, and 812. Particularly, step 80 includes setting t=1, and T=number of frames to evaluate. For every iteration of step 818, set t=1+1. Step 810 includes using a lowpass spatial filter or the lie using a Gaussian kernel, to smooth image t. Step 812 including, optionally, using a temporal filter to remove temporal noise in the image I*, etc. using a recursive filter in Eq (1).

Subsequent to image filtering, the method may proceed to detection and description of features at steps 814, 816, and 818. Step 814 includes setting t=1, setting T=number of frames to evaluate. For every iteration of 812, set t=1+1. Step 816 includes using a defined feature detector, detecting speckle in the image, and defining the center of each speckle as a feature. Step 818 includes using an area around features detected in 816, and describing each feature using spatial or frequency domain. Next, the method may proceed to feature tracking.

Features tracking may include steps 820, 822, 824, 826, 828, and 830. Step 820 includes setting t=1, and setting T=‘number of frames to evaluate’−1. For every iteration of step 830, t=t+1. Step 822 includes using features and feature descriptors from steps 816 and 818 to create a cost matrix to the difference between for any possible linking of Features from frame t and t+1. In steps 824, 826, and 828: for each element in the cost matrix in 822, if element i,j is above a threshold, set element i,j cost to infinite. Step 830 includes using an assignment algorithm using the costmatrix from step 822 to connect features between frame t and t+1. Next, the method may proceed to region tracking.

Region tracking may include steps 832, 834, 836, 838, 840, and 842. Step 832 includes setting t=1 and setting T=‘number of frames to evaluate’−1. For every iteration of step 842, set t=1+1. At step 834: For every X_{b}, drift compensation is implemented so that X_{b}(1)=X_{b}(T−1). Step s836 includes using Eq (4) such that each individual feature is normalized, so the sum of all the weight is equal to 1. Step 838 includes using Eq (5) such each individual feature is normalized, so the sum of all the weight is equal to 1. Step 840 includes using Eq (6) to calculate the next possible position of Y. Step 842 includes using a Kalman filter to update next position of Xb using Y as the measured position. Next, the method may return to step 806.

If “No” at step 806, the method proceeds to steps for strain estimation, which includes steps 844, 846, and 848. Step 844 includes setting z=1 and setting Z=arbitrary number depending on how many strain curves you wish, but Z<B. For every iteration of step 848, set z=1+1. Step 846 includes calculating the interest contour region (L) as the total distance between an arbitrary number<=B of coordinate points X_{b}(t) from step 842, for all frames 1<=t<=T. Step 848 includes using Eq (7) to calculate strain. After strain, the method may end.

The present disclosure addresses this shortcoming by providing, in part, systems and methods, and algorithms provided therein, for the estimation of cardiac strain using high frame rate ultrasound. The systems and methods provided herein are designed to analyze high frame rate ultrasound (HFR-US) images and estimate strain curves by tracking motion. In embodiments, the methods are accomplished by tracking small translations between frames using features and estimate strain curves along the myocardial wall contour. Further, it is possible to estimate both longitudinal and circumferential strain using the systems and methods provided here.

Accordingly, embodiments of the present disclosure provide methods for estimating strain/motion determination from high-speed 2D or 3D ultrasound images from a subject. An example, method may include: (a) obtaining a 2D or 3D ultrasound image; (b) filtering out high frequency temporal noise; (c) extracting features by identifying and extracting the largest pixel intensity within a 3×3 pixel neighborhood and using it as a descriptor; (d) tracking said features by linking individual features between frames using the feature descriptor; (e) combining the bulk displacement of feature tracks within a larger area into a continuous track that describes the myocardial displacement; (f) calculating the cardiac strain to describe the contraction between two points independent of overall cardiac motion; and (g) estimating strain/motion determination.

In some embodiments, the method further comprises administering to a subject an appropriate therapy based on the estimation.

According to embodiments of the present disclosure, systems and methods are provided for estimating strain/motion determination from high-speed 2D or 3D ultrasound images from a subject. The systems and methods are designed to analyze HFR-US images and estimate strain curves by tracking motion. This is done by tracking small translations between frames using features and estimate strain curves along the myocardial wall contour. Further, it is possible to estimate both longitudinal and circumferential strain using the provided systems and methods. In examples, there are five basic steps to performing the methods described herein. After obtaining high-speed 2D or 3D images from the subject, high frequency temporal noise is filtered out.

As ultrasound images contain a significant amount of information that does not relate to motion and make motion estimation difficult. High frequency temporal noise can be removed using a simple first order recursive filter to get a filtered image (Î(x,y,t)) by using the following equation.

Î(x,y,t)=αₜ×Î(x,y,t)+(1−αₜ)×I(x,y,t) (3)

where αₜ is an integration constant taking a value between 0 and 1, I(x,y,t) is the ultrasound image in frame t and Î(x,y,t−1) is the filtered image of the previous frame t−1. For this version αₜ=0.5. To improve the extraction of features a spatial filter is also used. A Gaussian spatial filter is implemented using a 3×3 pixel window. Because ultrasound sampling varies with respect to depth in Cartesian coordinates and is not as constant as in spherical coordinates the filter is implemented in spherical coordinates.

Next, features may be extracted by identifying and extracting the largest pixel intensity within a 3×3 pixel neighborhood and using it as a descriptor. When estimating motion one option is to only evaluate pixels in the images where there is a higher likelihood to estimate motion correctly. Feature extraction is based on finding particular pixels (p(ρ,θ,t)), where ρ is the scan depth, θ is the azimuth angle and t is the frame, which are best suited for motion detection.

Feature extraction is based on developing a suitable numeric description (Descriptor) in terms of a neighborhood of p(ρ,θ,t) to qualify the feature. There are multiple sophisticated descriptors available such as SURF, HOG or FREAK. But, if the translations are small sophisticated descriptors are not necessary as long as the frame to frame translation has a maximum limit. In this algorithm, the feature is defined by the largest pixel intensity within a 3×3 pixel neighborhood. The intensity of the particular pixel p(ρ,θ,t) is extracted and used as a descriptor.

Next, the features can then be tracked by linking individual features between frames using the feature descriptor. After extracting features, the features are tracked by linking individual features between frames using the feature descriptor. As this can be considered an assignment problem the Hungarian algorithm is implemented to assign features to feature tracks. The absolute intensity ratio between features in frame t and t−1 in decibel (dB) is used as the cost matrix. Furthermore if the intensity difference between features is above 6 dB or translation is above the maximum frame to frame translation (T×vₘₐₓ×1.25 where T is temporal resolution (T=1/FR) and vₘₐₓ is 240 mm/s which is the maximum velocity of a healthy myocardial wall as estimated by TDI) the cost of linking the two features will be set to infinity.

The bulk displacement of feature tracks are then combined within a larger area into a continuous track that describes the myocardial displacement. Out of plane motion and decorrelation of speckle that happens as a function of distance makes it improbable to track individual features through an entire cardiac cycle. Instead the bulk displacement of feature tracks within a larger area are combined into a continuous track that describe the myocardial displacement (Xₙ). The initial points {X₁(1), . . . , X_{B}(1)} and width of the myocardium is defined by manually marking the mid myocardium along the entire contour and the thickness region in the myocardium (L_{T}). An ellipsoid is fitted to the points and divided into arbitrary number of points (B) which are evenly divided with respect to angle where the right ventricular free wall is used as an anatomical reference. In order to estimate the bulk of displacement a weighted average is calculated for all feature tracks within a L_{T}/2 radius of Xₙ(t). First, a Gaussian weight (w_{G}) was calculated for each feature track, depending on the distance to Xₙ as defined by equation (4):

$${w}_{G} = \frac{1}{2 π σ} {e}^{\frac{{d ( {x}_{j} ( t ) , {x}_{n} ( t - 1 ) )}^{2}}{2 {σ}^{2}}} \qquad ( 4 )$$

where d(xⱼ(t),Xₙ(t−1)) is the Euclidean distance between the feature (xⱼ(t)) and Xₙ(t−1) and σ=\sqrt{L_{T}}. Second, the inverse directionality difference weight (w_{D}) of the feature tracks with respect to the bulk displacement of a 22 ms window (τ_{w}) as defined by equation:

$${w}_{D} ( j ) = \frac{1}{d ( \frac{d}{d {τ}_{w}} {x}_{j} ( t ) , ( t - 1 ) )} \qquad ( 5 )$$

where

$$\frac{d}{d {τ}_{w}} {x}_{j}$$

is the directional components of feature track j and

is the median of the directional components of all feature tracks in a specific region. w_{G} and w_{D} are both normalized so Σw_{G}=Σw_{D}=1. The estimated component (Y) is calculated using equation:

$$Y = {X}_{n} ( t - 1 ) + {\sum }_{j = 1}^{J} {x}_{j} ( t ) \times ( \frac{{α}_{G}}{{α}_{G} + {α}_{D}} \frac{d}{dt} \times {w}_{G} ( j ) + \frac{{α}_{D}}{{α}_{G} + {α}_{D}} \times {w}_{D} ( j ) ) \qquad ( 6 )$$

where α_{D} and α_{G} are the confidence in each of the weights. For this version α_{D}=α_{G}=1. Xₙ(t) is then updated recursively using using a second order Kalman model and Y as the new measured state. At the end of each cardiac cycle drift compensation is calculated by multiplying a constant variable at all times t′ where Xₙ(t′) have the opposite direction of the drift component.

The cardiac strain is then calculated to describe the contraction between two points independent of overall cardiac motion. Cardiac strain (ε(t)) is calculated to describe the contraction between two points independent of overall cardiac motion. ε(t) is the percentage change in distance with respect to an initial condition, which is defined by equation:

$$ɛ ( t ) = \frac{L ( t ) - L ( 1 )}{L ( 1 )} \times 100 \% \qquad ( 7 )$$

where L(t) is the Euclidean distance between multiple region tracks {X₁, . . . , X_{B}}. The strain/motion determination is then estimated.

In some embodiments, the method further comprises administering to a subject an appropriate therapy based on the estimation. Such therapy will be dependent on the estimation derived from the provided system and methods and can be readily determined by one skilled in the art.

The present subject matter may be a system, a method, and/or a computer program product. The computer program product may include a computer readable storage medium (or media) having computer readable program instructions thereon for causing a processor to carry out aspects of the present subject matter.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present subject matter may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present subject matter.

Aspects of the present subject matter are described herein with reference to flowchart illustrations and/or block diagrams of methods, apparatus (systems), and computer program products according to embodiments of the subject matter. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present subject matter. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While certain embodiments have been described, these embodiments have been presented by way of example only, and are not intended to limit the scope of the present disclosure. Indeed, the novel methods, devices, and systems described herein may be embodied in a variety of other forms. Furthermore, various omissions, substitutions, and changes in the form of the methods, devices, and systems described herein may be made without departing from the spirit of the present disclosure. The accompanying claims and their equivalents are intended to cover such forms or modifications as would fall within the scope and spirit of the present disclosure.

1. A method comprising:
receiving frames of images acquired from a subject over a period of time;
determining, in the frames of images, speckles of features of the subject;
tracking bulk, averaged movement of the speckles between image frames; and
determining displacement of at least a portion of the subject based on the tracked bulk movement.

2. The method of claim 1, wherein the frames of images are ultrasound images.

3. (canceled)

4. The method of claim 1, wherein the subject is a heart,
wherein determining speckles of features comprises determining speckles of a myocardial wall of the heart,
wherein determining displacement comprises determining displacement of the myocardial wall based on the tracked bulk movement.

5. The method of claim 1, wherein determining speckles of features comprises determining speckles of features of the subject in B-mode images.

6. The method of claim 1, wherein each speckle is defined by an intensity of a pixel of one of the frames of images, and a coordinate of the pixel.

7. The method of claim 6, wherein the intensity of each speckle is above a predetermined threshold for a region of interest in the frame.

8. The method of claim 1, wherein determining displacement comprises:
determining whether the determined displacement of suspected speckle exceeds a predetermined distance within another period of time; and
in response to determining that the determined displacement exceeds the predetermined distance within the other period of time, removing the suspected speckle as an outlier.

9. The method of claim 1, wherein determining displacement comprises averaging positions of speckles within a region of interest of two or more frames, wherein the determined displacement is the displacement of the averaged positions.

10. The method of claim 1, wherein determining displacement comprises:
determining a drift of the subject during a movement cycle; and
adjusting the determined displacement based on the determined drift.

11. The method of claim 10, wherein determining the drift comprises determining a drift of a heart during a cardiac cycle.

12. The method of claim 1, wherein the determined displacement represents a cardiac strain.

13. The method of claim 1, further comprising applying, to the images, image filtering in temporal and spatial domains.

14. The method of claim 1, further comprising using an ultrasound image acquisition system to acquire the frames of images.

15. The method of claim 1, wherein the at least a portion of the subject is at least a first portion of the subject, and
wherein the method further comprises:
determining displacement of at least a second portion of the subject based on the tracked bulk movement; and
determining a time ordering of the displacement of the at least a first portion of the subject and the at least a second portion of the subject; and
displaying an image that indicates the time ordering.

16. The method of claim 1, further comprising determining velocity of the at least a portion of the subject based on the displacement over the period of time.

17. The method of claim 1, further comprising acquiring the frames of images at a high frame rate.

18-34. (canceled)

35. A method comprising:
receiving frames of images acquired from a subject over a period of time;
qualifying, in the frames of images, speckles of features of the subject based on brightness;
tracking bulk movement of the qualified speckles between image frames; and
determining displacement of at least a portion of the subject based on the tracked bulk movement.

36. (canceled)

37. (canceled)

38. The method of claim 35, wherein the subject is a heart,
wherein determining speckles of features comprises determining speckles of a myocardial wall of the heart,
wherein determining displacement comprises determining displacement of the myocardial wall based on the tracked bulk movement.

39-41. (canceled)

42. The method of claim 35, wherein determining displacement comprises:
determining whether the determined displacement of suspected speckle exceeds a predetermined distance within another period of time; and
in response to determining that the determined displacement exceeds the predetermined distance within the other period of time, removing the suspected speckle as an outlier.

43-48. (canceled)

49. The method of claim 35, wherein the at least a portion of the subject is at least a first portion of the subject, and
wherein the method further comprises:
determining displacement of at least a second portion of the subject based on the tracked bulk movement; and
determining a time ordering of the displacement of the at least a first portion of the subject and the at least a second portion of the subject; and
displaying an image that indicates the time ordering.

50. The method of claim 35, further comprising determining velocity of the at least a portion of the subject based on the displacement over the period of time.

51-68. (canceled)
